(19) United States
(12) Patent Application Publication
(10) Pub. No.: US 2019/0170046 A1
(43) Pub. Date: Jun. 6, 2019

(54) CONVERSION OF CARBON DIOXIDE FROM VEHICLE EXHAUST TO LIQUID FUELS AND FUEL ADDITIVES

(71) Applicant: Saudi Arabian Oil Company, Dhahran (SA)

(72) Inventors: Esam Z. Hamad, Dhahran (SA); Husain A. Baaqel, Dhahran (SA)

(73) Assignee: Saudi Arabian Oil Company, Dhahran (SA)

(21) Appl. No.: 15/828,887

(22) Filed: Dec. 1, 2017

(51) Int. Cl. F01N 3/36; C07C 1/12; C25B 1/04; C07C 31/04

(52) U.S. Cl. CPC F01N 3/36; C07C 1/12; C10G 2400/04; C07C 31/04; C10G 2400/02; C25B 1/04

(57) ABSTRACT

Embodiments of a system for on-site conversion of carbon dioxide from vehicle exhaust to liquid fuels and fuel additives comprise a carbon dioxide collection system, an external power source, an electrolyzer, and a carbon dioxide conversion system. The carbon dioxide collection system interfaces with a mobile carbon dioxide capture system onboard a vehicle to transfer CO₂ captured from vehicle exhaust to a vessel in the carbon dioxide collection system. The external power source provides the energy required for operation of the carbon dioxide conversion system and the electrolyzer. The electrolyzer separates a water feed into hydrogen and oxygen to generate a hydrogen feed and an oxygen feed. The carbon dioxide conversion system converts the CO₂ collected from the exhaust of the vehicles and delivered to the carbon dioxide collection system and the hydrogen feed from the electrolyzer into useful liquid fuels and fuel additives through electrochemical reduction.

### BACKGROUND

### Field

Embodiments of the present disclosure generally relate to a carbon dioxide conversion system and, more specifically relate to a system for on-site conversion of carbon dioxide from vehicle exhaust to liquid fuels and fuel additives.

### Technical Background

Vehicles driving on roads and in factories throughout the world generate carbon dioxide as part of the exhaust from their propulsion systems. The carbon dioxide is generally formed as a waste product from the combustion of hydrocarbons in an internal combustion utilizing gasoline, diesel, or natural gas for example. The persistent release of carbon dioxide, considered a greenhouse gas, into the atmosphere is considered a contributing factor by scientists to increases in global temperatures. The ability to capture the carbon dioxide from a vehicle's exhaust and sequester it in an alternative form is considered desirable to reduce the release of carbon dioxide into the environment.

Carbon dioxide capture and conversion is a challenging process due to the energy intensity of conversion with carbon dioxide being a stable chemical. Presently the energy used in carbon dioxide conversion processes comes from fossil fuels. Utilization of fossil fuels to convert captured carbon dioxide is counterproductive to the original purpose of a carbon capture process. Specifically, burning carbon dioxide generating fossil fuels to convert captured carbon dioxide does not result in a net reduction in carbon dioxide expelled into the environment because of inefficiencies in the conversion process and the energy required to initially capture the carbon dioxide.

Accordingly, ongoing needs exists for efficient carbon capture and utilization where carbon dioxide conversion processes are environmentally green and produce fuels with sufficient thermal efficiency which can be utilized immediately.

### SUMMARY

Embodiments of the present disclosure are directed to a system for on-site conversion of carbon dioxide from vehicle exhaust to liquid fuels and fuel additives. Carbon dioxide captures from vehicle exhaust and stored on-board the emitting vehicle is delivered to a fueling station where it can be converted to a variety of fuel blends like octane enhances such as methanol and cetane enhanced such as dimethyl ether. The system may convert the collected CO₂ to only one type of fuel blend or more than one blend using multiple CO₂ conversion units. The fuels created may also be blended if necessary for optimum use and composition for varying vehicle types. As the conversion of the CO₂ is completed at the same site as refueling of the vehicle the system eliminates the need to transport captured CO₂ from the fueling stations for conversion and minimizes infrastructure needs for the mobile carbon dioxide capture.

According to one embodiment, a system for on-site conversion of carbon dioxide from vehicle exhaust to liquid fuels and fuel additives is provided. The system comprises a carbon dioxide collection system, an external power source, an electrolyzer, and a carbon dioxide conversion system. The carbon dioxide collection system interfaces with a mobile carbon dioxide capture system onboard a vehicle to transfer CO₂ captured from vehicle exhaust to a vessel in the carbon dioxide collection system. The external power source provides the energy required for operation of the carbon dioxide conversion system and the electrolyzer. The electrolyzer separates a water feed into hydrogen and oxygen to generate a hydrogen feed and an oxygen feed. The carbon dioxide conversion system converts the CO₂ collected from the exhaust of the vehicles and delivered to the carbon dioxide collection system and the hydrogen feed from the electrolyzer into useful liquid fuels and fuel additives through electrochemical reduction.

In a further embodiment, a further system for on-site conversion of carbon dioxide from vehicle exhaust to liquid fuels and fuel additives is provided. The system comprises a carbon dioxide collection system, an external power source, a carbon dioxide conversion system, and a liquid fuel blending system. The carbon dioxide collection system interfaces with a mobile carbon dioxide capture system onboard a vehicle to transfer CO₂ captured from vehicle exhaust to a vessel in the carbon dioxide collection system. The external power source provides the energy required for operation of the carbon dioxide conversion system. The carbon dioxide conversion system converts the CO₂ collected from the exhaust of the vehicles and delivered to the carbon dioxide collection into useful liquid fuels and fuel additives through electrochemical reduction. The liquid fuel blending system comprising one or more mixing units which combine the liquid fuels and fuel additives produced by the carbon dioxide conversion system in various ratios or combine one or more of the liquid fuels and fuel additives produced by the carbon dioxide conversion system with one or more traditional fossil fuels in various ratios.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a flow chart of a system for on-site conversion of carbon dioxide from vehicle exhaust to liquid fuels in accordance with one or more embodiments of the present disclosure.

FIG. 2 is a flow chart of a system for on-site conversion of carbon dioxide from vehicle exhaust to liquid fuels and fuel additives in accordance with one or more embodiments of the present disclosure.

FIG. 3 is a flow chart of a system for on-site conversion of carbon dioxide from vehicle exhaust to liquid fuels and fuel additives with an oxidation reactor in accordance with one or more embodiments of the present disclosure.

FIG. 4 is a reaction scheme illustrating an example series of oxidative chemical reactions to form a cetane boosting additive and octane boosting additive from toluene.

### DETAILED DESCRIPTION

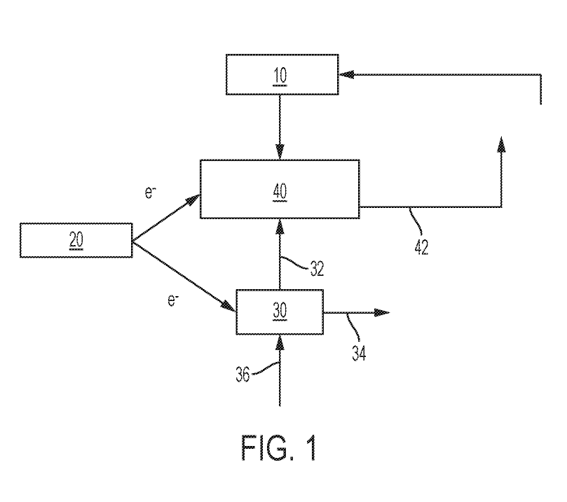

Reference will now be made in detail to embodiments of the system for on-site conversion of carbon dioxide from vehicle exhaust to liquid fuels and fuel additives of the present disclosure. Though the systems for on-site conversion of carbon dioxide from vehicle exhaust to liquid fuels and fuel additives of FIGS. 1, 2, and 3 are provided as exemplary, it should be understood that the present systems encompass other configurations.

The system for on-site conversion of carbon dioxide from vehicle exhaust to liquid fuels and fuel additives aims to convert compressed captured CO₂ from vehicles into fuels and blending components on-site at the location of collection of the captured CO₂ and the fueling station. A synergy is provided where carbon dioxide processed in the system for on-site conversion of carbon dioxide from vehicle exhaust to liquid fuels and fuel additives is captured from mobile sources to reduce the carbon footprint of the mobile sources, but is then also utilized and converted to high value liquid fuels. The system for on-site conversion of carbon dioxide from vehicle exhaust to liquid fuels and fuel additives can take energy from non-fossil sources such as solar and wind and store the collected energy in the form of high energy liquid fuels. By collecting carbon dioxide from vehicles via mobile collection and converting the carbon dioxide to liquid fuels at a fueling station the system for on-site conversion of carbon dioxide from vehicle exhaust to liquid fuels and fuel additives eliminates the need to secondarily transport captured carbon dioxide to a conversion plant. The carbon dioxide is delivered by the vehicle concurrently with the vehicle filling its fuel tank with liquid fuel generated at the same location.

With reference to FIG. 1, the system for on-site conversion of carbon dioxide from vehicle exhaust to liquid fuels and fuel additives includes a carbon dioxide collection system 10, an external power source 20, an electrolyzer 30, and a carbon dioxide conversion system 40. A mobile carbon dioxide capture system captures CO₂ on board a vehicle from an exhaust stream of the vehicle and delivers it to a fuel station and the carbon dioxide collection system 10. The CO₂ captured in the mobile carbon dioxide capture system is delivered to the carbon dioxide collection system 10 for utilization in the system for on-site conversion of carbon dioxide from vehicle exhaust to liquid fuels and fuel additives. The external power source 20 provides the energy required for operation of the carbon dioxide conversion system 40 and the electrolyzer 30. The electrolyzer 30 provides the carbon dioxide conversion system 40 with a hydrogen feed 32 from the splitting of water in a water feed 36 to the electrolyzer 30 into its constituent parts of hydrogen and water. Utilizing compressed CO₂ from the carbon dioxide collection system 10, the hydrogen feed 32 from the electrolyzer 30, and energy from the external power source 20, the carbon dioxide conversion system 40 generates useful fuels that can be used in a variety of different vehicles and engine types.

The conversion of CO₂ may be done either by unloading it at a fuel station while fueling the vehicle, specifically fueling and unloading CO₂ concurrently or sequentially, then transporting it to a larger centralized conversion plant or by converting it at the fuel station if the area allows for fitting such technology. Converting the CO₂ at the fuel station will reduce transportation costs and emissions resulting from the fuel burned to transport the fuels to a conversion plant.

The mobile carbon dioxide capture system may be any system affixed to or integrated with a vehicle's exhaust system configured to capture CO₂ from the vehicle exhaust stream. The specific configuration and mechanisms for CO₂ capture, collection, and storage on-board the vehicle are outside the scope of this disclosure. Non-limiting examples of mobile carbon dioxide capture systems are provided in U.S. Pat. No. 9,175,591 issued on Nov. 3, 2015 and directed to a Process and System Employing Phase-Changing Absorbents and Magnetically Responsive Sorbent Particles for On-Board Recovery of Carbon Dioxide from Mobile Sources, the contents of which are incorporated by reference. Further non-limiting examples of mobile carbon dioxide capture systems are provided in U.S. Pat. No. 9,180,401 issued on Nov. 10, 2015 and directed to a Liquid, Slurry and Flowable Powder Adsorption/Absorption Method and System Utilizing Waste Heat for On-Board Recovery and Storage of CO₂ from Motor Vehicle Internal Combustion Engine Exhaust Gases, the contents of which are incorporated by reference.

In one or more embodiments the carbon dioxide collection system 10 interfaces with the mobile carbon dioxide capture system to transfer CO₂ captured from vehicle exhaust to a vessel in the carbon dioxide collection system 10. The interface may be any transfer mechanism and configuration known to one skilled in the art. For example, CO₂ may be transferred via a pressurized hose connected to ports on the carbon dioxide collection system 10 and the reservoir of the mobile carbon dioxide capture system. The transfer mechanism for unloading the CO2 to the carbon dioxide collection system 10 from the reservoir of the mobile carbon dioxide capture system may be the same or similar to those utilized for filling natural gas in a compressed natural gas (CNG) engine vehicle as both systems are configured for transfer of compressed gases. Additionally, safety measures utilized for filling natural gas in a CNG engine vehicle may also be implemented in the transfer between the carbon dioxide collection system 10 and the reservoir of the mobile carbon dioxide capture system.

In one or more embodiments the carbon dioxide collection system 10 comprises a CO₂ storage vessel for storage of compressed CO₂. The CO₂ storage vessel may be located at the fueling station, the actual CO₂ conversion plant, or at least one CO₂ storage vessel at each location. It will be appreciated that the CO₂ storage vessel may be sized according to the demands of the carbon dioxide conversion system 40 and the volume of CO₂ deposited by the mobile carbon dioxide capture systems. The CO₂ may be retained in the CO₂ storage vessel at an elevated pressure. In various embodiments, the pressure in the CO₂ storage vessel is sufficiently high to retain the CO₂ in a liquid form. CO₂ forms a liquid at approximately 860 pounds per square inch (psi) or 58.5 atmosphere (atm) at 72° F. (22.2° C.). To ensure the CO₂ maintains its liquid state. In various embodiments the pressure in the CO₂ storage vessel may range from 100 to 300 bar at ambient temperatures.

In one or more embodiments, the external power source 20 provides the energy required for operation of the carbon dioxide conversion system 40 and the electrolyzer 30. The external power source 20 provides the energy to power the conversion of the CO₂ collected in the mobile carbon dioxide capture system and delivered to the carbon dioxide collection system 10 to liquid fuels and fuel additives. In one or more embodiments the external power source 20 comprises non-fossil energy to provide power to the carbon dioxide conversion system 40, the electrolyzer 30, or both. Examples of non-fossil energy used on one or more embodiments include wind power from an on-site wind power generator, solar power from an on-site photovoltaic array, or hydroelectric power from an on-site hydroelectric generator.

In one or more embodiments the electrolyzer 30 separates a water feed into hydrogen and oxygen to generate a hydrogen feed 32 and an oxygen feed 34 through an electrolysis process. Specifically, electrolysis of water is the decomposition of water into oxygen and hydrogen gas as a result an electric current being passed through the water. In practice in the electrolyzer 30, a DC current from the external power source 20 is connected to two electrodes, or two plates which are placed in the water. The electrodes or plates are typically made from an inert metal such as platinum, stainless steel or iridium. Hydrogen appears at the cathode electrode or plate where electrons enter the water and oxygen appears at the anode electrode or plate. Assuming ideal faradaic efficiency, the amount of hydrogen generated is twice the amount of oxygen, and both are proportional to the total electrical charge conducted by the solution. The hydrogen feed 32 is provided to the carbon dioxide conversion system 40 for utilization in the conversion CO₂ to useful liquid fuels and fuel additives.

In pure water at the negatively charged cathode, a reduction reaction takes place, with electrons (e) from the cathode being given to hydrogen cations to form hydrogen gas. The half reaction at the cathode is in accordance with reaction (1).

2H⁺(aq)+2e⁻→H₂(g) (1)

Similarly, at the positively charged anode, an oxidation reaction occurs, generating oxygen gas and giving electrons to the anode to complete the circuit in accordance with reaction (2).

2H₂O(l)→O₂(g)+4H⁺(aq)+4e⁻ (2)

The overall reaction when the two half reactions are combined produces 2 molecules of hydrogen gas (H₂) and one molecule of oxygen gas (O₂) from every two molecules of water (H₂O) in accordance with reaction (3).

2H₂O(l)→2H₂(g)+O₂(g) (3)

The electrolysis reaction of water into hydrogen and water has a standard potential of −1.23 V, meaning it ideally requires a potential difference of 1.23 volts to split the water. However, electrolysis of pure water requires excess energy in the form of overpotential to overcome various activation barriers. Without the excess energy the electrolysis of pure water occurs very slowly or not at all due to the limited self-ionization of water. The efficiency of the electrolyzer 30 may be increased through the addition of an electrolyte such as a salt, an acid or a base and the use of electrocatalysts.

The carbon dioxide conversion system 40 performs the actual conversion of the CO₂ collected from the exhaust of vehicles and delivered to the carbon dioxide collection system 10 into useful liquid fuels and fuel additives 42. The carbon dioxide conversion system 40 operates in accordance with any known chemical conversion of CO₂ to liquid fuels or fuel additives 42 known to one skilled in the art. In one or more embodiments, the CO₂ is converted to fuels and fuel additives 42 in a 2-step process. Specifically, hydrogen is produced from water through electrolysis in the electrolyzer 30 in a first step, then using the H₂ as a feed to the carbon dioxide conversion system 40 to produce fuels 42 from the CO₂ in the second step. Systems and processes for converting H₂ and CO₂ to useful fuels 42 are known to those skilled in the art. Any known process for converting H₂ and CO₂ to useful fuels 42 may be utilized in the system for on-site conversion of carbon dioxide from vehicle exhaust to liquid fuels and fuel additives of the present disclosure.

The carbon dioxide conversion system 40 may utilize a catalyst to drive the electrochemical reduction of CO₂ to liquid fuels and fuel additives 42. In various embodiments, the catalysts used for the electrochemical reduction of CO₂ include metal macrocycles such as Ni(I) and Ni(II) macrocycles, Co(I) tetraaza macrocycles, Pd complexes, Ru(II) complexes, and Cu(II) complexes. To produce an organic peroxide a catalyst such as N-Hydroxyphthalimide may be utilized. To produce an alcohol or aldehyde a two catalyst system such as N-Hydroxyphthalimide and Cobalt or similar metal may be utilized.

The electrochemical reduction of CO₂ may generate a variety of products. Some products are spontaneously generated and other products require input of additional energy to drive the reaction. As a general rule, the Gibbs Free energy (ΔG °) must be negative for the reaction to spontaneously occur at constant temperature and pressure. Similarly, the standard potential (e) must be positive for the reaction to spontaneously occur at constant temperature and pressure. The only CO₂ reactions that are spontaneous are reactions with metal oxides or metal hydroxides to form metal carbonates, and some reactions with high energy molecules such as peroxides. Table 1 provides the Gibbs Free energy and standard potential for various electrochemical reductions of CO₂. A non-spontaneous reaction requires energy input to increase the Gibbs energy of the product compared to the reactants.

TABLE 1
Gibbs Free energy and standard potential
for electrochemical reduction of CO₂

| Reaction | ΔG⁰ (kJ/mol) | E⁰ |
|---|---|---|
| CO₂ + e⁻ → CO₂⁻ | 183.32 | −1.90 |
| CO₂ + 2H⁺ + 2e⁻ → CO + H₂O | 19.88 | −0.10 |
| CO₂ + 2H⁺ + 2e⁻ → HCOOH | 38.40 | −0.20 |
| CO₂ + 6H⁺ + 6e⁻ → CH₃OH + H₂O | −17.95 | 0.03 |
| CO₂ + 8H⁺ + 8e⁻ → CH₄ + 2H₂O | −130.40 | 0.17 |
| 2CO₂ + 12H⁺ + 12e⁻ → C₂H₄ + 4H₂O | −40.52 | 0.07 |
| 2CO₂ + 12H⁺ + 12e⁻ → C₂H₅OH + 3H₂O | −49.21 | 0.085 |
| 3CO₂ + 18H⁺ + 18e⁻ → C₃H₇OH + 5H₂O | −52.1 | 0.09 |

In further embodiments, CO₂ can also be converted into liquid fuels 42 in a single step process where water and CO₂ are used directly. That is the electrolyzer 30 is omitted from the system for on-site conversion of carbon dioxide from vehicle exhaust to liquid fuels and fuel additives and water and CO₂ are fed directly to the carbon dioxide conversion system 40. For example, the electrochemical conversion of CO₂ into ethanol using a copper nanoparticle/n-doped graphene electrode completes the transformation in a single step process. Such a single step process is detailed in Yang Song, et al., “High-Selectivity Electrochemical Conversion of CO₂ to Ethanol using a Copper Nanoparticle/N-Doped Graphene Electrode” ChemistrySelect 2016, 1, 1-8 which is incorporated by reference in its entirety. In this process, CO₂ and water are used as reactants in a fuel cell where an electrochemical reaction takes place to produce ethanol directly.

The carbon dioxide conversion system 40 converts H₂ and CO₂ or water and CO₂ to useful fuels and fuel additives 42. A variety of fuels 42 may be formed with uses as both fuels directly as well as octane or cetane enhancers for mixture with conventional fuels. The Research Octane Number (RON) is used to measure the resistance of fuels to auto-ignition and is an important specification for internal combustion engines. Table 2 provides the properties for a variety of formed liquid fuels as well as the high-level synthesis procedure and use.

TABLE 2
Example Liquid Fuels and Fuel Additives

| | Boiling | | | | | | |
|---|---|---|---|---|---|---|---|
| | Point | | Δ_{c}H° | Δ_{f}G° | Synthesis | Synthesis | |
| Product | (° C.) | RON | (MJ/L) | (kJ/mol) | Materials | Conditions | Example Use |
| Dimethyl | −24 | 35 | −23.3 | −112.9 | Catalyst | Reaction at | Fuel blend |
| Ether | | (55-60 | | | to reduce | less than | with Diesel |
| | | CN) | | | CO₂ | 200° C. | and Cetane |
| | | | | | | | enhancer |
| Methanol | 64.7 | 110-116 | −17.9 | −166.2 | Catalyst | Reaction at | Octane |
| | | | | | and | 125-165° C. | enhancer |
| | | | | | Ethereal | | |
| | | | | | solvent | | |
| Ethanol | 78.37 | 113 | −22.3 | −174.1 | Catalyst | Reaction at | Octane |
| | | | | | and water | Room | enhancer |
| | | | | | (2-step | Temperature | |
| | | | | | process) | | |
| Propanol | 97 | 109-118 | −24 | −170.7 | Catalyst, | Reaction at | Octane |
| | | | | | C₂H₄, | approximately | enhance |
| | | | | | and H₂ | 200° C. | |
| Ethylene | −103.7 | N/A | −28.576 | 68.1 | Catalyst | Reaction at | Compressed |
| | | | | | to reduce | Room | Natural Gas |
| | | | | | CO₂ | temperature | Fuel and |
| | | | | | | | energy |
| | | | | | | | Carrier |
| Methane | −161.5 | N/A | −23.5 | 50.6 | Catalyst | Reaction at | Compressed |
| | | | | | to reduce | less than | Natural Gas |
| | | | | | CO₂ | 160° C. | Fuel |

Which specific liquid fuels and fuel additives 42 are formed from the collected CO₂ may be determined at the fuel station level. For example, the options for producing dimethyl ether, methanol, or both may be made at the fuel depot which collects the CO₂ and generates the liquid fuel and fuel additives 42. A given catalyst generally produces a single species out of all the potential liquid fuels and fuel additives capable of being produced with the carbon dioxide conversion system 40. In one or more embodiments, the system for on-site conversion of carbon dioxide from vehicle exhaust to liquid fuels and fuel additives may include a single carbon dioxide conversion system 40 with a single catalyst capable of producing a single fuel or fuel additive 42. In further embodiments, the system for on-site conversion of carbon dioxide from vehicle exhaust to liquid fuels and fuel additives may include a multiple carbon dioxide conversion systems 40, each with a single catalyst capable of producing a single fuel or fuel additive 42, to allow for the production of multiple liquid fuels and fuel additives concurrently. It will be appreciated that a single carbon dioxide conversion system 40 may also include multiple catalysts which are selectable to generate different liquid fuels and fuel additives 42a/42b based on present demand and supply available at the fueling station.

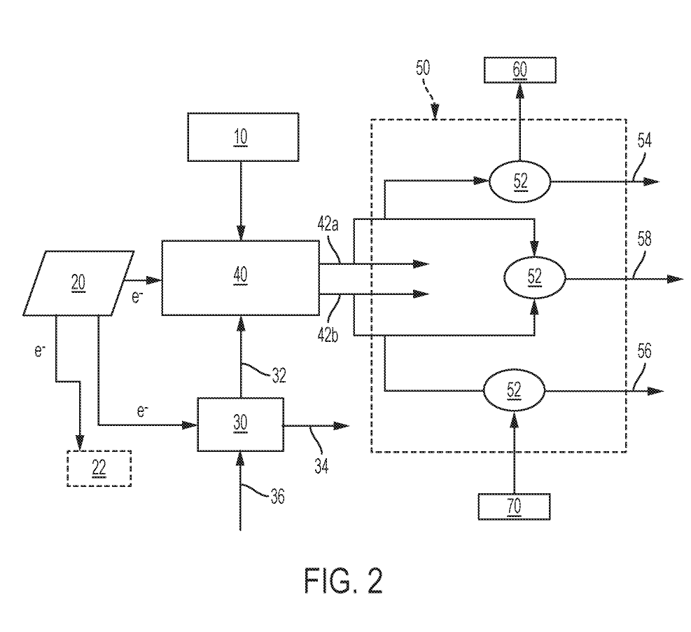

With reference to FIG. 2, in one or more embodiments, the system for on-site conversion of carbon dioxide from vehicle exhaust to liquid fuels and fuel additives further includes a liquid fuel blending system 50. The liquid fuel blending system 50 comprises one or more mixing units 52 which combine the products of the carbon dioxide conversion system 40 in various ratios or combine one or more of the products of the carbon dioxide conversion system 40 with one or more traditional fossil fuels in various ratios. The products of the carbon dioxide conversion system 40 are the liquid fuels and fuel additives 42. For example, in one or more embodiments, one or more products of the carbon dioxide conversion system 40 are mixed with diesel fuel from a diesel fuel reservoir 60 to produce a high-cetane diesel 54. Specifically, dimethyl ether from the carbon dioxide conversion system 40 may be mixed with the diesel fuel to produce the high-cetane diesel 54. Further, in one or more embodiments, one or more products of the carbon dioxide conversion system 40 are mixed with gasoline from a gasoline reservoir 70 to produce a high-octane gasoline 56. Specifically, methanol from the carbon dioxide conversion system 40 may be mixed with the gasoline to produce the high-octane gasoline 56. A mid-octane liquid fuel 58 may also be formed by mixing the dimethyl ether and the methanol from the carbon dioxide conversion system 40 in various ratios. The ratios of dimethyl ether and methanol included in the final blend may vary based on the standards and specifications specific to the region where the blend is used. For example, in Europe, the current maximum oxygen content in the blend should not exceed 3.7 wt % (11 wt % dimethyl ether or 7.4 wt % methanol) assuming the blend contains only one of the components. Similarly, the current oxygen specification in the United States is 2.7 wt % (8 wt % dimethyl ether and 5.4% methanol). So the range can be anywhere between 0% and the max specification wt % set by the regulatory authorities in that region.

In one or more embodiments, the oxygen feed 34 produced by the electrolyzer 30 from the electrolysis of water to generate the hydrogen feed 32 for the carbon dioxide conversion system 40 is utilized to convert low octane components into high octane components for mixture with the liquid fuels. For example, low octane components such as paraffins may be converted into high octane components such as alcohols, ketones, and aldehydes using partial oxidation. Similarly, high cetane components may be formed such as peroxides.

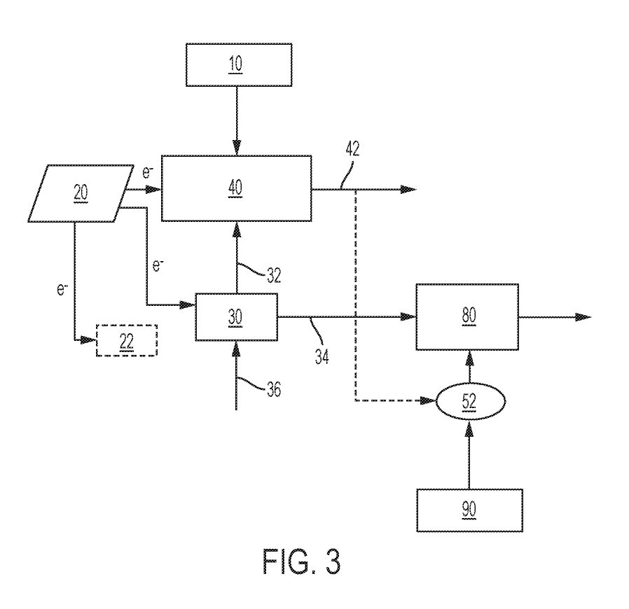

With reference to FIG. 3, the system for on-site conversion of carbon dioxide from vehicle exhaust to liquid fuels and fuel additives may include an oxidation reactor 80 to oxidize original fuels 90, liquid fuels generated by the carbon dioxide conversion system 40, or a mixture of both into products with a greater octane or greater cetane. For purposes of this disclosure the term “original fuels” means hydrocarbons introduced directly into the system and not the products of the carbon dioxide conversion system 40. The original fuels may be provided from a refinery or similar plant outside the carbon dioxide conversion system 40. The oxidation reactor 80 may receive oxygen in the oxygen feed 34 from the electrolyzer 30 to oxidize feed streams of fuel to alcohols, aldehydes, ketones, peroxides, and other converted products which would be known to those skilled in the art. The hydrocarbons fed to the oxidation reactor 80 may comprise original fuels 90 such as naptha provided as a raw feed source, a mixture of one or more liquid fuels generated by the carbon dioxide conversion system 40, or a mixture of both.

Table 3 provides some examples of generic Octane and Cetane enhancers which may be formed from the oxidation of fuel streams. The oxidation reactor 80 provides the added benefit of utilizing the waste oxygen produced by the electrolyzer 30 in generating the hydrogen feed 32 from water for the carbon dioxide conversion system 40 and in the process generating increased octane or increased cetane enhanced quality fuels. The enhanced quality fuels generated in the oxidation reactor 80 may be stored and utilized separately from the liquid fuels generated by the carbon dioxide conversion system 40 or may be mixed and combined in various ratios to generate a multitude of fuel products to meet fueling demands of various engine types.

TABLE 3
Octane and Cetane enhancers formed from oxidizing fuels

| | | Synthesis | |
|---|---|---|---|
| Product | Synthesis Materials | Conditions | Example uses |
| Organic | Hydrocarbon, O₂ and | Reaction at | Cetane enhancer |
| peroxide | Catalyst (example: N- | less than | |
| | Hydroxyphthalimide) | 100° C. | |
| Alcohols and | Hydrocarbon, O₂ and | Reaction at | Octane enhancer |
| Aldehydes | two catalysts (example: | less than | |
| | N-Hydroxyphthalimide | 100° C. | |
| | and Cobolt - or similar | | |
| | metal) | | |

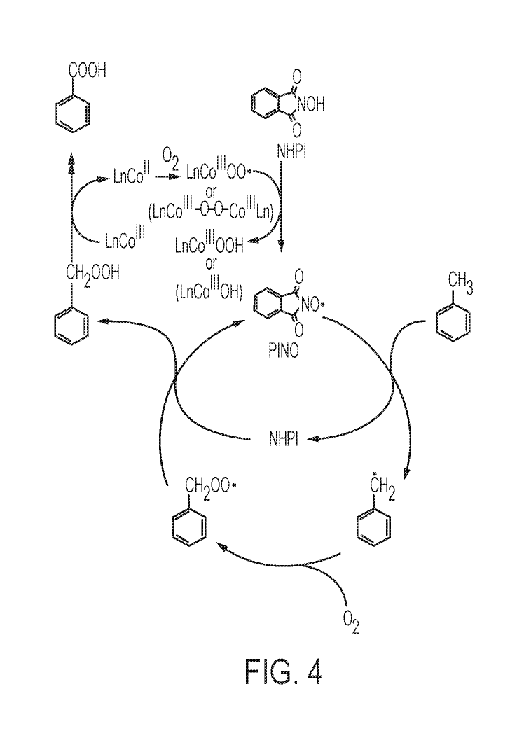

With reference to FIG. 4, and example scheme for generation of octane and cetane enhancers is provided. Specifically, FIG. 4 provides the scheme for how toluene may be oxidized to generate benzyl hydroperoxide as a cetane boosting additive and subsequently benzoic acid as an octane boosting additive. The scheme also provides example catalysts which may be utilized to accomplish each step of the transformation.

The oxidation reaction produced in the oxidation reactor 80 is an exothermic reaction. The thermal energy released by the exothermic reaction in the oxidation reactor 80 may be utilized to reduce the energy demands of the external power source 20. The thermal energy generated from the exothermic reactions may be utilized directly to heat feed streams to the carbon dioxide conversion system 40 or the electrolyzer 30. The heat generated from the exothermic reactions in the oxidation reactor 80 can also be used directly in the chemical conversion of CO₂ in reactions that require heat for initiation to avoid the need for alternative supplemental heat. Similarly, the thermal energy generated from the exothermic reactions may be utilized indirectly to operate a generator to generate electrical power to augment the external power source 20. Electricity may also be generated using devices for waste heat recovery such as thermoelectrics or using the Rankine cycle.

In one or more embodiments, the oxygen from the electrolyzer 30 is retained in an oxygen reservoir (not shown) and is provided to vehicles when the vehicles are off-loading collected CO₂ from the mobile carbon dioxide capture system which captured CO₂ on board the vehicle from the exhaust stream of the vehicle, fueling the vehicle with liquid fuels generated in the system for on-site conversion of carbon dioxide from vehicle exhaust to liquid fuels and fuel additives, or both. The vehicle may then oxidize fuels onboard with an on-board oxidation system (not shown) to produce increased cetane or octane fuels.

In one or more embodiments, the system for on-site conversion of carbon dioxide from vehicle exhaust to liquid fuels and fuel additives also includes a battery 22 electrically connected to the external power source 20. The battery 22 may collect surplus electrical energy from the external power source 20 during times when the carbon dioxide conversion system 40 and the electrolyzer 30 do not utilize the entirety of the power generated by the external power source 20. In one or more embodiments, the battery 22 may directly power the carbon dioxide conversion system 40 and the electrolyzer 30 with the external power source 20 continuously recharging the battery 22. In further embodiments, the external power source 20 may power the carbon dioxide conversion system 40 and the electrolyzer 30 during times of operation and the battery 22 is only charged during pauses in the operation of the carbon dioxide conversion system 40 and the electrolyzer 30. The battery 22 for storage of electrical energy is especially advantageous when the external power source 20 has variability or intermittency in the ability to generate power. For example, wind power generation may vary based on time of time, meteorological conditions, or other variables which affect wind speeds and direction and consequently affect power generation. Similarly, solar power generation may vary based on time of day, the solar calendar, meteorological condition, or other variable which affect the strength, position, and duration of solar energy reaching the photovoltaic cells. Even hydroelectric power generation may experience variability in power generation based on variability in flow rates as a result of drought conditions reducing release of water through the hydroelectric generators.

### Arithmetic Examples

The formation of the liquid fuels and fuel additives from non-fossil fuel sources may be validated as feasible arithmetically. Specifically, the raw materials and energy required to process CO₂ captured from the exhaust of a vehicle and convert the same to a variety of liquid fuels and fuel additives may be calculated. Assuming 60% of CO₂ is captured on-board the vehicle and is delivered to the carbon dioxide collection system 10, each vehicle would provide approximately 137 kilograms (kg) or 3113 moles of CO₂ per fueling cycle. Further assuming 100% conversion of the captured CO₂ to liquid fuels where Δ_{f}G ° for CO₂ is −394.39 and for H₂O is −237.14 kJ/mol, the energy required for conversion to specific liquid fuels and fuel additives may be determined.

It should now be understood the various aspects of the system for on-site conversion of carbon dioxide from vehicle exhaust to liquid fuels and fuel additives are described and such aspects may be utilized in conjunction with various other aspects.

In a first aspect, the disclosure provides a system for on-site conversion of carbon dioxide from vehicle exhaust to liquid fuels and fuel additives. The system comprises a carbon dioxide collection system, an external power source, an electrolyzer, and a carbon dioxide conversion system. The carbon dioxide collection system interfaces with a mobile carbon dioxide capture system onboard a vehicle to transfer CO₂ captured from vehicle exhaust to a vessel in the carbon dioxide collection system. The external power source provides the energy required for operation of the carbon dioxide conversion system and the electrolyzer. The electrolyzer separates a water feed into hydrogen and oxygen to generate a hydrogen feed and an oxygen feed. The carbon dioxide conversion system converts the CO₂ collected from the exhaust of the vehicles and delivered to the carbon dioxide collection system and the hydrogen feed from the electrolyzer into useful liquid fuels and fuel additives through electrochemical reduction.

In a second aspect, the disclosure provides the system of the first aspect, in which the system further comprises a liquid fuel blending system, the liquid fuel blending system comprising one or more mixing units which combine the liquid fuels and fuel additives produced by the carbon dioxide conversion system in various ratios or combine one or more of the liquid fuels and fuel additives produced by the carbon dioxide conversion system with one or more traditional fossil fuels in various ratios.

In a third aspect, the disclosure provides the system of the first or second aspects, in which one or more products of the carbon dioxide conversion system are mixed with diesel fuel to produce a high-cetane diesel.

In a fourth aspect, the disclosure provides the system of the third aspect, in which wherein dimethyl ether from the carbon dioxide conversion system is mixed with diesel fuel to produce the high-cetane diesel.

In a fifth aspect, the disclosure provides the system of any of the first through fourth aspects, in which one or more products of the carbon dioxide conversion system are mixed with gasoline to produce a high-octane gasoline.

In a sixth aspect, the disclosure provides the system of the fifth aspect, in which wherein methanol from the carbon dioxide conversion system is mixed with gasoline to produce the high-octane gasoline.

In a seventh aspect, the disclosure provides the system of any of the first through sixth aspects, in which dimethyl ether and methanol from the carbon dioxide conversion system are mixed to form a mid-octane liquid fuel.

In an eighth aspect, the disclosure provides the system of any of the first through seventh aspects, in which external power source comprises non-fossil energy.

In a ninth aspect, the disclosure provides the system of the eighth aspect, in which the external power source comprises one or more of an on-site wind power generator, an on-site photovoltaic array, or an on-site hydroelectric generator.

In a tenth aspect, the disclosure provides the system of any of the first through ninth aspects, in which the carbon dioxide conversion system utilizes a catalyst to drive the electrochemical reduction of CO₂ to liquid fuels and fuel additives.

In an eleventh aspect, the disclosure provides the system of the tenth aspect, in which the catalysts used for the electrochemical reduction of CO₂ comprises one or more of metal macrocycles, Pd complexes, Ru(II) complexes, and Cu(II) complexes.

In a twelfth aspect, the disclosure provides the system of any of the first through eleventh aspects, in which the system further comprises an oxidation reactor configured to oxidize original fuels, liquid fuels generated by the carbon dioxide conversion system, or a mixture of both into products with a greater octane or greater cetane.

In a thirteenth aspect, the disclosure provides the system of any of the twelfth aspect, in which the oxidation reactor utilizes the oxygen feed generated in the electrolyzer as an oxidizing agent to oxidize the original fuels, the liquid fuels and fuel additives generated by the carbon dioxide conversion system, or the mixture of both into products with a greater octane or greater cetane.

In a fourteenth aspect, the disclosure provides the system of the twelfth or thirteenth aspects, in which thermal energy released by the oxidation of fuels in the oxidation reactor is utilized to reduce the energy demands of the external power source.

In a fifteenth aspect, the disclosure provides the system of the fourteenth aspect, in which the thermal energy released by the oxidation of fuels in the oxidation reactor is utilized directly in the carbon dioxide conversion system for the chemical conversion of CO₂ in reactions that require heat for initiation to reduce or eliminate the need for alternative supplemental heat.

In a sixteenth aspect, the disclosure provides the system of the fourteenth or fifteenth aspects, in which the thermal energy released by the oxidation of fuels in the oxidation reactor is utilized indirectly to operate a generator to generate electrical power to augment the external power source.

In a seventeenth aspect, the disclosure provides a system for on-site conversion of carbon dioxide from vehicle exhaust to liquid fuels and fuel additives. The system comprises a carbon dioxide collection system, an external power source, a carbon dioxide conversion system, and a liquid fuel blending system. The carbon dioxide collection system interfaces with a mobile carbon dioxide capture system onboard a vehicle to transfer CO₂ captured from vehicle exhaust to a vessel in the carbon dioxide collection system. The external power source provides the energy required for operation of the carbon dioxide conversion system. The carbon dioxide conversion system converts the CO₂ collected from the exhaust of the vehicles and delivered to the carbon dioxide collection into useful liquid fuels and fuel additives through electrochemical reduction. The liquid fuel blending system comprising one or more mixing units which combine the liquid fuels and fuel additives produced by the carbon dioxide conversion system in various ratios or combine one or more of the liquid fuels and fuel additives produced by the carbon dioxide conversion system with one or more traditional fossil fuels in various ratios.

In an eighteenth aspect, the disclosure provides the system of the seventeenth aspect, in which one or more products of the carbon dioxide conversion system are mixed with diesel fuel to produce a high-cetane diesel.

In a nineteenth aspect, the disclosure provides the system of the eighteenth aspect, in which dimethyl ether from the carbon dioxide conversion system is mixed with diesel fuel to produce the high-cetane diesel.

In a twentieth aspect, the disclosure provides the system of any of the seventeenth through nineteenth aspects, in which one or more products of the carbon dioxide conversion system are mixed with gasoline to produce a high-octane gasoline.

In a twenty-first aspect, the disclosure provides the system of the twentieth aspect, in which methanol from the carbon dioxide conversion system is mixed with gasoline to produce the high-octane gasoline.

In a twenty-second aspect, the disclosure provides the system of the any of the seventeenth through twenty-first aspects, in which dimethyl ether and methanol from the carbon dioxide conversion system are mixed to form a mid-octane liquid fuel.

In a twenty-third aspect, the disclosure provides the system of any of the seventeenth through twenty-second aspects, in which the external power source comprises non-fossil energy.

In a twenty-fourth aspect, the disclosure provides the system of the twenty-third aspect, in which the external power source comprises one or more of an on-site wind power generator, an on-site photovoltaic array, or an on-site hydroelectric generator.

In a twenty-fifth aspect, the disclosure provides the method of any of the seventeenth through twenty-fourth aspects, in which the system further comprises an oxidation reactor configured to oxidize original fuels, liquid fuels and fuel additives generated by the carbon dioxide conversion system, or a mixture of both into products with a greater octane or greater cetane.

In a twenty-sixth aspect, the disclosure provides the method of the twenty-fifth aspect, in which thermal energy released by the oxidation of fuels in the oxidation reactor is utilized to reduce the energy demands of the external power source.

In a twenty-seventh aspect, the disclosure provides the method of the twenty-sixth aspect, in which the thermal energy is utilized directly in the carbon dioxide conversion system for the chemical conversion of CO₂ in reactions that require heat for initiation to reduce or eliminate the need for alternative supplemental heat.

In a twenty-eighth aspect, the disclosure provides the method of the twenty-sixth or twenty-seventh aspects, in which the thermal energy is utilized indirectly to operate a generator to generate electrical power to augment the external power source.

It should be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure of the claimed subject matter and should be interpreted in like manner as the more commonly used open-ended preamble term “comprising.”

What is claimed is:

1. A system for conversion of carbon dioxide from vehicle exhaust to liquid fuels and fuel additives,
the system comprising:
a carbon dioxide collection system,
an external power source,
an electrolyzer, and
a carbon dioxide conversion system, wherein
the carbon dioxide collection system interfaces with a mobile carbon dioxide capture system onboard a vehicle to transfer CO₂ captured from vehicle exhaust to a vessel in the carbon dioxide collection system;
the external power source provides the energy required for operation of the carbon dioxide conversion system and the electrolyzer;
the electrolyzer separates a water feed into hydrogen and oxygen to generate a hydrogen feed and an oxygen feed; and
the carbon dioxide conversion system converts the CO₂ collected from the exhaust of the vehicles and delivered to the carbon dioxide collection system and the hydrogen feed from the electrolyzer into useful liquid fuels and fuel additives through electrochemical reduction.

2. The system of claim 1 wherein the system further comprises a liquid fuel blending system, the liquid fuel blending system comprising one or more mixing units which combine the liquid fuels and fuel additives produced by the carbon dioxide conversion system in various ratios or combine one or more of the liquid fuels and fuel additives produced by the carbon dioxide conversion system with one or more traditional fossil fuels in various ratios.

3. The system of claim 2 wherein one or more products of the carbon dioxide conversion system are mixed with diesel fuel to produce a high-cetane diesel.

4. The system of claim 2 wherein one or more products of the carbon dioxide conversion system are mixed with gasoline to produce a high-octane gasoline.

5. The system of claim 2 wherein dimethyl ether and methanol from the carbon dioxide conversion system are mixed to form a mid-octane liquid fuel.

6. The system of claim 1 wherein the external power source comprises non-fossil energy.

7. The system of claim 1 wherein the carbon dioxide conversion system utilizes a catalyst to drive the electrochemical reduction of CO₂ to liquid fuels and fuel additives.

8. The system of claim 1 wherein the system further comprises an oxidation reactor configured to oxidize original fuels, liquid fuels generated by the carbon dioxide conversion system, or a mixture of both into products with a greater octane or greater cetane.

9. The system of claim 8 wherein the oxidation reactor utilizes the oxygen feed generated in the electrolyzer as an oxidizing agent to oxidize the original fuels, the liquid fuels and fuel additives generated by the carbon dioxide conversion system, or the mixture of both into products with a greater octane or greater cetane.

10. The system of claim 8 wherein thermal energy released by the oxidation of fuels in the oxidation reactor is utilized to reduce the energy demands of the external power source.

11. The system of claim 10 wherein the thermal energy is utilized directly in the carbon dioxide conversion system for the chemical conversion of CO₂ in reactions that require heat for initiation to reduce or eliminate the need for alternative supplemental heat.

12. The system of claim 10 wherein the thermal energy is utilized indirectly to operate a generator to generate electrical power to augment the external power source.

13. A system for conversion of carbon dioxide from vehicle exhaust to liquid fuels and fuel additives, the system comprising:
a carbon dioxide collection system,
an external power source,
a carbon dioxide conversion system, and
a liquid fuel blending system, wherein
the carbon dioxide collection system interfaces with a mobile carbon dioxide capture system onboard a vehicle to transfer CO₂ captured from vehicle exhaust to a vessel in the carbon dioxide collection system;
the external power source provides the energy required for operation of the carbon dioxide conversion system;
the carbon dioxide conversion system converts the CO₂ collected from the exhaust of the vehicles and delivered to the carbon dioxide collection into useful liquid fuels and fuel additives through electrochemical reduction; and
the liquid fuel blending system comprising one or more mixing units which combine the liquid fuels and fuel additives produced by the carbon dioxide conversion system in various ratios or combine one or more of the liquid fuels and fuel additives produced by the carbon dioxide conversion system with one or more traditional fossil fuels in various ratios.

14. The system of claim 13 wherein one or more products of the carbon dioxide conversion system are mixed with diesel fuel to produce a high-cetane diesel.

15. The system of claim 13 wherein one or more products of the carbon dioxide conversion system are mixed with gasoline to produce a high-octane gasoline.

16. The system of claim 13 wherein dimethyl ether and methanol from the carbon dioxide conversion system are mixed to form a mid-octane liquid fuel.

17. The system of claim 13 wherein the external power source comprises non-fossil energy.

18. The system of claim 17 wherein the external power source comprises one or more of an on-site wind power generator, an on-site photovoltaic array, or an on-site hydroelectric generator.

19. The system of claim 13 wherein the system further comprises an oxidation reactor configured to oxidize original fuels, liquid fuels and fuel additives generated by the carbon dioxide conversion system, or a mixture of both into products with a greater octane or greater cetane.

20. The system of claim 19 wherein thermal energy released by the oxidation of fuels in the oxidation reactor is utilized to reduce the energy demands of the external power source.
